(19) United States
(12) Patent Application Publication
(10) Pub. No.: US 2009/0192448 A1
(43) Pub. Date: Jul. 30, 2009

(54) ORAL GASTRIC LAVAGE APPARATUS

(76) Inventor: Anthony R. Talamonti, Kenosha, WI (US)

Correspondence Address: QUARLES & BRADY LLP, 411 E. WISCONSIN AVENUE, SUITE 2040, MILWAUKEE, WI, 53202-4497, US

(21) Appl. No.: 12/361,096

(22) Filed: Jan. 28, 2009

Related U.S. Application Data
(60) Provisional application No. 61/024,349, filed on Jan. 29, 2008.

(51) Int. Cl. A61M 1/00

(52) U.S. Cl. 604/35

(57) ABSTRACT

A oral gastric lavage apparatus includes a tube that has a longitudinal first lumen and a smaller longitudinal second lumen. The tube has a proximate end with a first opening for connecting a source of suction to the first lumen and a second opening for connecting an irrigant source to the second lumen. The first lumen as plurality of inlets near a distal end of the tube and the second lumen as a plurality of outlets near the distal end. A hydrophilic coating is applied to the outer surface of the tube. A tapered tip is attached to distal end of the tube and is formed of a material that is softer and more flexible than the material of the tube.

### CROSS-REFERENCE TO RELATED APPLICATIONS

This application claims benefit of U.S. Provisional Patent Application No. 61/024,349 filed on Jan. 29, 2008.

### STATEMENT REGARDING FEDERALLY SPONSORED RESEARCH OR DEVELOPMENT

Not Applicable

### BACKGROUND OF THE INVENTION

1. Field of the Invention

The present invention relates to a medical lavage apparatus and more particularly to an apparatus used for oral gastric lavage.

2. Description of the Related Art

Acute poisoning is a common cause of morbidity and mortality in children and adults. However, if ingested poison can be removed from the gastrointestinal track before being absorbed, the risk of severe poisoning is reduced. One method of removing ingested poison is that of oral gastric lavage in which the gastrointestinal track or stomach is successively irrigated and aspirated through a lavage tube inserted along the patient's gastrointestinal track to the stomach.

A common method of oral gastric lavage is described in U.S. Pat. No. 5,667,500 which uses parallel connected syringe cylinders having plungers and valves to allow both irrigation and aspiration through a single nozzle connected to a single lumen pliable lavage tube. Such a system requires continual, manual pumping by an attendant.

An improvement is taught in U.S. Pat. No. 5,890,516 which provides a double lumen flexible oral gastric lavage tube, a first lumen delivers an irrigation liquid and the second lumen is used for aspiration through an in-wall vacuum system. The exterior wall of the lavage tube defines a larger first lumen used for aspiration and a smaller circular second lumen is within and to one side of the first lumen adjacent the exterior wall. In some procedures, the relatively small size of the second lumen limited the delivery rate of the irrigation liquid to less that the desired level.

Conventional oral gastric lavage tubes are made of polyvinyl chloride (PVC) which provides a flexible tube that yet is sufficiently rigid to prevent collapsing when the vacuum is applied. However, PVC resists being slid into the esophagus. To overcome that resistance, a lubricant, such as K-Y® jelly (registered trademark of Johnson & Johnson Corporation, New Brunswick, N.J., USA), was manually applied to the oral gastric lavage tube by medical personal prior to insertion into a patient. This added a step which delayed insertion and also lubricated the gloved hands of the person applying the lubricant making it difficult for that person to insert the oral gastric lavage tube.

Another drawback of an oral gastric lavage tube made of relatively stiff PVC is the possibility that during insertion, the tip of the tube may scratch the mucus membranes of the esophagus. Prior attempts to overcome this drawback involved forming the remote end of the PVC tube into a curved tip. Even with the curvature, the remote end was relatively stiff.

### SUMMARY OF THE INVENTION

A oral gastric lavage apparatus comprises a tube having a first lumen and a second lumen. A first connector is adapted for attaching a source of suction to the first lumen and a second connector is adapted for attaching a source of an irrigant to the second lumen, wherein both connectors are near a proximate end of the tube. A plurality of first apertures provide paths between the first lumen and the exterior of the tube and a plurality of second apertures provide other paths between the second lumen and the exterior of the tube. The first and second apertures are near a distal end of the tube. The tube has an outer surface with a hydrophilic coating thereon. In one embodiment of the oral gastric lavage apparatus, the hydrophilic coating is polyvinyl pyrrolidone baked onto the outer surface.

In a preferred embodiment of the oral gastric lavage apparatus, a stripe of radiaopaque material extends along the length of a portion of the tube that is intended for insertion into a patient.

Another aspect of the oral gastric lavage apparatus is a tip at the distal end of the tube, wherein the tip of made of a material that is that is softer and more flexible than the material of the tube. Preferably the tip contains a radiaopaque material.

### BRIEF DESCRIPTION OF THE DRAWINGS

FIG. 1 is a perspective view of components of an oral gastric lavage kit that includes a tube according to the present invention;

FIG. 2 illustrates the features of the oral gastric lavage tube in the kit;

FIG. 3 is a transverse cross sectional view through the oral gastric lavage tube along line 3-3 in FIG. 2; and

FIG. 4 is a longitudinal cross sectional view of a remote end of the oral gastric lavage tube.

### DETAILED DESCRIPTION OF THE INVENTION

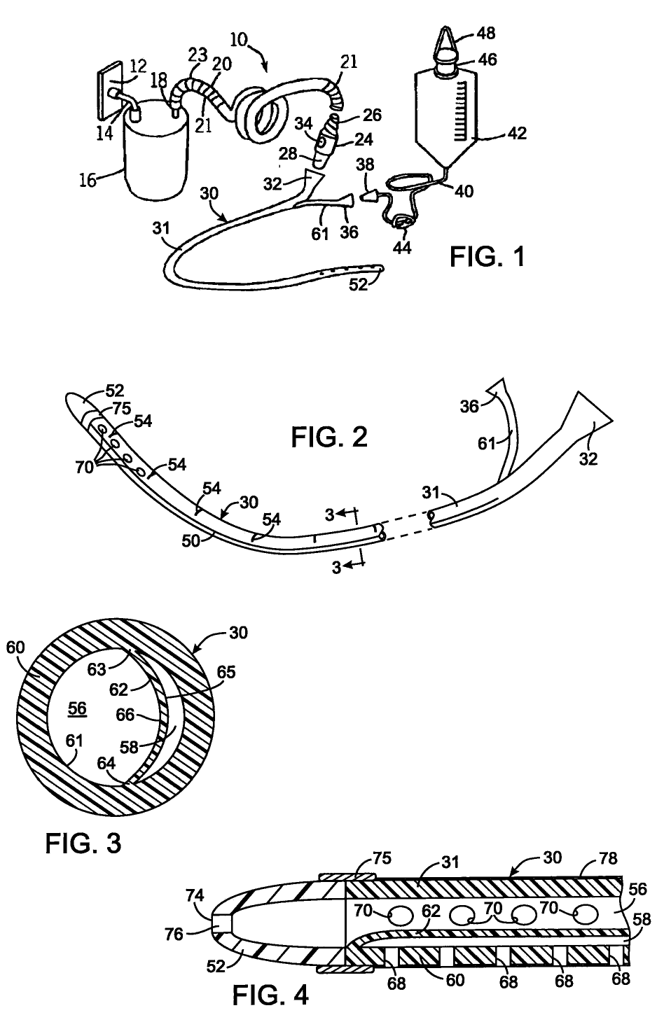

With initial reference to FIG. 1, an oral gastric lavage kit 10 is adapted for use with an in-wall vacuum system 12 of a medical facility from which a vacuum line 14 is coupled to a collection vessel 16 to draw air there from. The collection vessel 16 serves as a trap for solids and liquids drawn through an inlet 18.

The oral gastric lavage kit 10 includes a vacuum tube 20 that is connected to the collection vessel inlet 18 provide a flexible source of suction for medical procedures. The vacuum tube 20 has an internal diameter that is greater than the opening of the inlet 18 as it fits over the outside of the inlet. A corrugated elbow 23 is used on the distal end of the vacuum tube 20 to prevent kinking. The corrugated elbow 23 may include a vortex device, such as a “corkscrew” spiral, made by an internal groove causing a vortex flow of liquid in the vacuum tube 20 near the inlet 18. The vortex device improves the flow of air and liquid.

The oral gastric lavage kit 10 further includes an aspiration valve 24 connected to a vacuum tube inlet 21 to provide upstream control of the vacuum pressure to a level less than that present at the vacuum tube 20. As described in U.S. Pat. No. 5,890,516, the aspiration valve 24 has a bleed-air inlet 34 allowing ingress of air to reduce the suction drawn on the oral gastric lavage tube 30. The ingress air flow is controlled by variably occluding an bleed-air inlet 34. This conventional aspiration valve 24 provides a step-tapered end 26 receiving the vacuum tube 20 by interference fit of the inner diameter of the vacuum tube against an outer ridged surface of the step-tapered end.

An opposite tapered end 28 of the aspiration valve 24 receives an outwardly flared, first connector collar 32 at the proximal end of an oral gastric lavage tube 30. That proximal end also has an outwardly flared, second connector collar 36 for receiving a corresponding cone connector 38 of an irrigation line 40 attached to a 3500 cc irrigation bag 42. The irrigation line 40 has a ratchet clamp 44 for metering flow of a conventional irrigation liquid, called an irrigant. The irrigation bag 42 provides a cap 46 and hanger 48 for suspension on an IV pole according to techniques well known in the art. As will be described, the oral gastric lavage tube 30 has two lumens one connected to the vacuum tube 20 for aspiration and another lumen connected to the irrigation line 40.

Referring to FIG. 2, the oral gastric lavage tube 30 has a distal end with a rounded tip 52. A radiaopaque stripe 50 extends from proximate the tip 52 substantially its entire length of tube, at least that portion of the length of the tube which typically is inserted into the patient. The radiaopaque stripe 50 assists in positioning the lavage tube 30 under fluoroscopic examination of the patient. The oral gastric lavage tube 30 also includes gradations 54 at regular distances, e.g., ten centimeters, along its length. Numbers may be associated with the gradations 54 to indicate the distance that a particular gradation is from the tip 52 to aid the medical personnel in determining the distance that the tube has been inserted into a patient and also observe motion of the tube into and out of the patient's mouth. Additional gradations may be provided at smaller distances.

With reference to FIG. 3, the interior of the oral gastric lavage tube 30 is divided into longitudinally extending first and second lumens 56 and 58, respectively. The two lumens 56 and 58 are separated by an interior wall 62 that extends in a curved manner between opposite sides of the inner circumferential surface 61 of the outer wall 60 of the oral gastric lavage tube 30, thereby forming a crescent shaped second lumen 58. The interior wall 62 joins the inner circumferential surface 61 at points 63 and 64 spaced apart 100 to 180 degrees around that surface. The interior wall 62 has a convex surface 65 within the smaller second lumen 58 and an opposite concave surface 66 within the larger first lumen 56. The curved configuration enables the interior wall 62 to resist deforming when vacuum is applied to the first lumen 56 and when irrigant flows through the second lumen 58.

The first lumen 56 is used for aspiration and communicates with the first connector collar 32 at the proximal end of the oral gastric lavage tube 30, as shown in FIG. 2. The second lumen 58 is smaller than the first lumen and is used for irrigation. For that purpose, the second lumen 58 communicates via a lateral tube 59 with the second connector collar 36. It should be understood that it is desirable to have a significantly larger lumen for aspiration in order to carry particles, such as food and undissolved pill fragments from the patient's stomach to the collection vessel 16. Since the second lumen 58 is utilized for irrigation and carries the liquid irrigant, it does not have to accommodate particles and thus can be smaller than the first lumen. Nevertheless, the second lumen must be sufficiently large to convey the irrigant at a desirable flow rate.

The first and second lumens 56 and 58 extend to the distal end of the oral gastric lavage tube 30 that is shown in FIG. 4. Near that distal end, the tube's interior wall 62 terminates by joining the outer wall 60 to close the second lumen 58 from the first lumen 56. A plurality of first apertures 68 extend through the outer wall 60 providing means for the irrigant introduced via the second connector collar 36 to exit the second lumen 58. Preferably there are five first apertures 68. A plurality of second apertures 70 extend from the first lumen 56 through the outer wall 60 of the oral gastric lavage tube 30. Preferably there are eight second apertures 70 arranged longitudinally in two lines each having four apertures and spaced apart approximately 120 degrees around the outer circumference of the main tube 31. One line of four second apertures 70 is visible in FIG. 2, and the other line of second apertures 70 can be seen in FIG. 4. The size of the second apertures 70 is no greater than the smallest orifice through which the aspirated material must flow to reach the collection vessel 16. Thus, particles cannot enter the oral gastric lavage tube 30 which could clog the system downstream.

The distal end of the oral gastric lavage tube 30 terminates at a soft tip 52 that is a separate piece from the PVC main tube 31. The tip 52, which preferably is fabricated of silicone, is considerably softer and more flexible than PVC, however the tip 52 also could be made of latex rubber. This forms a deformable and non-abrasive distal tip of the oral gastric lavage tube 30. The tip 52 curves to a blunted region 74 that has a smaller diameter than the outer diameter of the main tube 31 and an aperture 76 extends through that blunted region 74. The tip 52 may be formed by molding, during which process the silicone is impregnated with a radiaopaque material, such as barium sulfate. An external band 75 is used to fasten the silicone tip 52 to the end of the main tube 31 with an adhesive, such as Dow Corning® 3145 adhesive available from the Dow Corning Corporation of Midland, Mich., USA.

With continuing reference to FIG. 4, the outer surface of the main tube 41 and the tip 52 has a baked-on hydrophilic coating 78 that becomes lubricious when made wet, such as when water is applied prior to insertion into a patient or when contacted by liquids in the esophagus. For example, this hydrophilic coating 78 may be polyvinyl pyrrolidone (PVP), such as is manufactured by ARMM, Inc., Huntington Beach, Calif., USA. This outer coating 78 enables the PVC main tube 41 to slide down the esophagus easily with little friction or resistance as compared to an uncoated PVC tube.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. A oral gastric lavage apparatus comprising:
a tube having a first lumen and a second lumen with a first connector adapted for attaching a source of suction to the first lumen and a second connector adapted for attaching a source of an irrigant to the second lumen, a plurality of first apertures communicating with the first lumen and extending through an outer wall of the tube and a plurality of second apertures communicating with the second lumen and extending through the outer wall, the tube having an outer surface with a hydrophilic coating thereon.

2. The oral gastric lavage apparatus as recited in claim 1 wherein the hydrophilic coating is polyvinyl pyrrolidone.

3. The oral gastric lavage apparatus as recited in claim 1 wherein the hydrophilic coating is baked on the outer surface of the tube.

4. The oral gastric lavage apparatus as recited in claim 1 further comprising a stripe of radiaopaque material extending longitudinally along the tube.

5. The oral gastric lavage apparatus as recited in claim 1 further comprising gradations at regular distances longitudinally along at least a portion of the tube.

6. The oral gastric lavage apparatus as recited in claim 1 wherein each of the plurality of first apertures is smaller than every orifice through which material flows from the plurality of first apertures to a collection vessel that is part of the source of suction.

7. The oral gastric lavage apparatus as recited in claim 1 wherein the tube is formed of polyvinyl chloride.

8. The oral gastric lavage apparatus as recited in claim 1 further comprising a tip attached to an end of the tube, wherein the tip is formed of a material that is softer and more flexible than the material of the tube.

9. The oral gastric lavage apparatus as recited in claim 8 wherein the tip is formed of a material selected from the group consisting of a silicone and a latex rubber.

10. The oral gastric lavage apparatus as recited in claim 1 further comprising a separate tip attached to an end of the tube, wherein the separate tip contains a radiaopaque material.

11. The oral gastric lavage apparatus as recited in claim 1 wherein the first and second lumens are separated by an interior wall that extends in a curved manner between opposite sides of the outer wall of the tube, wherein the interior wall has convex surface within the second lumen and an opposite concave surface within the first lumen.

12. A oral gastric lavage apparatus comprising:
a tube having a first lumen and a second lumen wherein the first lumen has a larger cross sectional area than the second lumen, and the tube having a proximate end section with a first opening for providing a path between the first lumen and a source of suction and the proximate end section also having a second opening for providing a path between the second lumen and a source of an irrigant, a plurality of first apertures communicating with the first lumen and extending through an outer wall of the tube and a plurality of second apertures communicating with the second lumen and extending through the outer wall, the tube having an outer surface with a hydrophilic coating thereon; and
a tapered tip attached to a distal end of the tube, wherein the tip is formed of a material that is softer and more flexible than the material of the tube.

13. The oral gastric lavage apparatus as recited in claim 12 wherein the hydrophilic coating is polyvinyl pyrrolidone.

14. The oral gastric lavage apparatus as recited in claim 12 wherein the hydrophilic coating is baked on the outer surface of the tube.

15. The oral gastric lavage apparatus as recited in claim 12 further comprising a stripe of radiaopaque material extending longitudinally along the tube.

16. The oral gastric lavage apparatus as recited in claim 12 further comprising gradations at regular distances longitudinally along at least a portion of the tube.

17. The oral gastric lavage apparatus as recited in claim 12 wherein each of the plurality of first apertures is smaller than every passage through which material flows from the plurality of first apertures to a collection vessel that is part of the source of suction.

18. The oral gastric lavage apparatus as recited in claim 12 wherein the tip is formed of a material selected from the group consisting of a silicone and a latex rubber.

19. The oral gastric lavage apparatus as recited in claim 12 wherein the tip contains a radiaopaque material.

20. The oral gastric lavage apparatus as recited in claim 12 wherein the first and second lumens are separated by an interior wall that extends in a curved manner between opposite sides of the outer wall of the tube, wherein the interior wall has convex surface within the second lumen and an opposite concave surface within the first lumen.
